(19) United States
(12) Patent Application Publication
(10) Pub. No.: US 2024/0299626 A1
(43) Pub. Date: Sep. 12, 2024

(54) BIODEGRADABLE POLYMER SUPPORT CONTAINING BIOACTIVE MATERIAL AND MANUFACTURING METHOD THEREFOR

(71) Applicant: CHA UNIVERSITY INDUSTRY-ACADEMIC COOPERATION FOUNDATION, Pocheon-si (KR)

(72) Inventors: Dong Keun HAN, Seoul (KR); Kyoung-Won KO, Yongin-si (KR); So-Yeon PARK, Seongnam-si (KR)

(73) Assignee: CHA UNIVERSITY INDUSTRY-ACADEMIC COOPERATION FOUNDATION, Pocheon-si (KR)

(21) Appl. No.: 18/549,437

(22) PCT Filed: Mar. 8, 2022
(86) PCT No.: PCT/KR2022/003267

§ 371 Date: Sep. 7, 2023

(30) Foreign Application Priority Data
Mar. 8, 2021 (KR) 10-2021-0030041

(51) Int. Cl. A61L 27/44; A61L 27/10; A61L 27/36; A61L 27/54; A61L 27/56; A61L 27/58; A61L 31/00; A61L 31/02; A61L 31/12; A61L 31/14; A61L 31/16

(52) U.S. Cl. CPC A61L 27/446; A61L 27/10; A61L 27/3633; A61L 27/54; A61L 27/56; A61L 27/58; A61L 31/005; A61L 31/026; A61L 31/128; A61L 31/146; A61L 31/148; A61L 31/16; A61L 2300/258; A61L 2400/18

(57) ABSTRACT

Provided are a biodegradable polymer scaffold including bioactive materials and methods of manufacturing the same. The biodegradable polymer scaffold, preventing inflammatory responses caused by acidic substances produced during a degradation process, has easily controllable mechanical strength, and includes bioactive materials derived from cells of target tissues, and thus may induce tissue regeneration more effectively.

### TECHNICAL FIELD

This application claims the benefit of Korean Patent Application No. 10-2021-0030041 filed on Mar. 8, 20201, in the Korean Intellectual Property Office, the disclosure of which is incorporated herein in its entirety by reference.

The present disclosure relates to a biodegradable polymer scaffold containing a bioactive material and a method of manufacturing the same.

### BACKGROUND ART

Tissue engineering, one of the new fields that have emerged with the development of science, is a multidisciplinary study that integrates and applies basic concepts and technologies of bioscience, biotechnology, and medical science, and is intended to maintain, improve, and restore the functions of human bodies by understanding the correlation between the structure and function of living tissue, and furthermore, by designing implantable artificial tissue to replace injured tissues or organs with normal tissues or regenerate injured tissues or organs.

A representative tissue engineering technology is summarized as follows. First, cells are isolated from the tissue after necessary tissue is collected from the body of a patient, and the isolated cells are proliferated by cultivation to a required amount. A hybrid cell/polymer structure obtained by loading the proliferated cells into a porous biodegradable polymer scaffold and culturing the cells in vitro for a certain period of time is implanted back into human bodies. Implanted cells are supplied with oxygen and nutrients by diffusion of body fluids until new blood vessels are formed in most tissues and organs. Once blood vessels grow in the body and blood is supplied, cells proliferate and differentiate to form new tissues and organs while the polymer scaffold is degraded and removed therefrom according to this method.

For such tissue engineering research, first, it is important to manufacture a biodegradable polymer scaffold similar to living tissue. The main requirements of a scaffold material used for regeneration of human tissues are sufficient cell affinity such that tissue cells adhere to the surface of the material and form tissue having a three-dimensional structure and acting as an intermediate barrier between implanted cells and host cells. This indicates that the scaffold material should have nontoxic biocompatibility without causing blood coagulation or inflammatory responses after implantation. In addition, the scaffold material should be biodegradable such that the scaffold material degrades while new tissue is formed and is ultimately removed therefrom without foreign substances remaining in the body. However, in general, biodegradable synthetic polymers have poor physical properties and acidic substances are produced during biodegradation thereof, causing problems of inflammatory responses and cytotoxicity in human bodies. In addition, natural polymers are limited to chemical modification and various applications have not been made due to poor physical and mechanical properties, difficulty in controlling period of degradation, and low solubility in organic solvents,

Therefore, there is a need to develop a highly functional composite scaffold capable of effectively inducing regeneration of target organs by improving biocompatibility and tissue regeneration ability, while simultaneously inhibiting inflammatory responses of cells and tissues by neutralizing acidic substances that are degradation products of a biodegradable polymer.

### DISCLOSURE

### Technical Problem

Provided is a biodegradable polymer scaffold including basic ceramic nanoparticles, an extracellular matrix, a bioactive material, and a biodegradable polymer. Provided is a biomedical implant including the biodegradable polymer scaffold.

Provided is a method of manufacturing a biodegradable polymer scaffold including: preparing basic ceramic nanoparticles; preparing a first polymer solution including the basic ceramic nanoparticles, an extracellular matrix, a bioactive material, and a biodegradable polymer; preparing a second polymer solution by mixing 100 to 2000 parts by weight of a pore inducer inducing pores having a size of 100 to 500 μm with 100 parts by weight of the first polymer solution; and preparing a porous polymer scaffold by freeze-drying the second polymer solution.

### Technical Solution

According to an aspect of the present disclosure, a biodegradable polymer scaffold includes basic ceramic nanoparticles, an extracellular matrix, a bioactive material, and a biodegradable polymer. For the biodegradable polymer scaffold, not only pore size, density, porosity of the porous polymer scaffold may be controlled, but also a shape and size of the polymer scaffold including the extracellular matrix and the bioactive material (DNA fragment mixture and extracellular vesicle) may be controlled in various ways. In addition, by including the basic ceramic nanoparticles, the extracellular matrix, and the bioactive material in various concentrations,

Inflammation inhibition ability, tissue regeneration ability, and hydrophilicity of the biodegradable polymer scaffold may be improved, and mechanical strength and a period of degradation may be controlled.

The basic ceramic nanoparticles may be selected from the group consisting of an alkali metal or an oxide or hydroxide thereof or an alkali earth metal or an oxide or hydroxide thereof. The alkali metal or alkali earth metal may be, for example, lithium (Li), beryllium (Be), sodium (Na), magnesium (Mg), potassium (K), calcium (Ca), rubidium (Rb), strontium (Sr), barium (Ba), cesium (Cs), francium (Fr), or radium (Ra). In addition, the oxide or hydroxide of the alkali metal or alkali earth metal may be, for example, lithium hydroxide, beryllium hydroxide, sodium hydroxide, magnesium hydroxide, potassium hydroxide, calcium hydroxide, rubidium hydroxide, strontium hydroxide, barium hydroxide, cesium hydroxide, francium hydroxide, radium hydroxide, magnesium oxide, sodium oxide, lithium oxide, sodium oxide, manganese oxide, potassium oxide, calcium oxide, barium oxide, cesium oxide, or radium oxide.

In an embodiment, the basic ceramic nanoparticles may be surface-modified with a fatty acid or a polymer. The fatty acid may be, for example, caprylic acid, capric acid, lauric acid, myristoleic acid, palmitoleic acid, sapienic acid, oleic acid, ellidic acid, vaccenic acid, linoleic acid, ricinoleic acid, linoleadic acid, a-linoleic acid, arachidonic acid, eicosapentaenoic acid, erucic acid, stearic acid, DHA, octatecanoic acid, coconut oil, palm oil, cotton oil, horse bud oil, soybean oil, olive oil, corn oil, sunflower oil, safflower oil, hemp oil, or canola oil. In addition, the polymer may be, for example, poly-L-lactide, poly-D-lactide, poly-D,L-lactide, polyglycolide, polycaprolactone, poly-L-lactide-co-glycolide, poly-D-lactide-co-glycolide, poly-D,L-lactide-co-glycolide, poly-L-lactide-co-caprolactone, poly-D-lactide-co-caprolactone, poly-D,L-lactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyphosphazene, polyanhydride, polysebacic anhydride, polyhydroxyalkanoate, polyhydroxybutylate, or polycyanoacrylate, which are formed of at least one monomer selected from the group consisting of L-lactide, D-lactide, D,L-lactide, glycolide, caprolactone, dioxanone, trimethylene carbonate, hydroxyalkanoate, peptide, cyanoacrylate, lactic acid, glycolic acid, hydroxycaproic acid, maleic acid, phosphazene, amino acid, hydroxybutylic acid, sebacic acid, hydroxyethoxyacetic acid, and trimethylene glycol. The biodegradable polymer scaffold including the basic ceramic nanoparticles surface-modified with the fatty acid or the polymer according to an embodiment may have improved dispersion stability in an organic solvent and enhanced mechanical properties and may also alleviate inflammatory responses and cytotoxicity in living bodies by neutralizing acidic substances with the basic ceramic particles.

In another embodiment, the basic ceramic nanoparticles or surface-modified basic ceramic nanoparticles may have a diameter of 1 nm to 1 mm. When the diameter of the basic ceramic nanoparticles exceeds the above-described range, precipitation may occur due to the weight of the basic ceramic nanoparticles causing a problem of phase separation in an organic solvent.

In another embodiment, the basic ceramic nanoparticles may be contained therein in an amount of 5 to 50 parts by weight based on 100 parts by weight of the polymer scaffold. The basic ceramic nanoparticles may be included in an amount of, for example, 5 to 50 parts by weight, 5 to 45 parts by weight, 5 to 40 parts by weight, 5 to 30 parts by weight, 10 to 50 parts by weight, 10 to 40 parts by weight, 10 to 30 parts by weight, 20 to 40 parts by weight, or 10 to 25 parts by weight, based on 100 parts by weight of the polymer scaffold. In this regard, when the amount of the basic ceramic nanoparticles is less than the above-described range, acidic substances that are degradation products of the polymer scaffold cannot be sufficiently neutralized. When the amount of the basic ceramic nanoparticles is greater than the above-described range, alkalization of an environment surrounding the polymer scaffold may be induced.

The extracellular matrix refers to substrate proteins derived from tissues or cells of humans or animals in a complexly mixed state or in artificially separated single molecule states, and the proteins may have a denatured structure. For example, the extracellular matrix may be derived from vertebrates such as humans, pigs, cows, rats, sheep, horses, dogs, or cats and may be separated from kidney, amnion, skin, small intestinal submucosa, fascia, or spinal cord membrane according to the intended use thereof. The extracellular matrix may be appropriately selected according to structure or function thereof. In an embodiment, the extracellular matrix may be a group having a fibrous structure, a group associated with osteogenic differentiation and osteogenesis, a glucosaminoglycan group, or a proteoglycan group. The group having a fibrous structure may be, for example, collagen fibers, elastin fibers, laminin, fibrinogen, fibronectin, gelatin, and the like. In this regard, the collagen may be type I, II, III, IV, V, VI, VII, VIII, IX, X, XI, XII, XIV, XV, XVI, XVIII, XIX, XX, XXI, XXII, XXIII, XXIV, XXV, XXVI, XXVII, or XXVIII collagen. In addition, the group associated with osteogenic differentiation and osteogenesis may be, for example, osteonectin, osteopontin, vitronectin, and vimentin. In addition, the glucosaminoglycan group may be, for example, heparan sulfate, keratan sulfate, chondroitin sulfate, dermatan sulfate, heparin, low molecular weight heparin, or hyaluronic acid. In addition, the proteoglycan group may be, for example, decorin, biglycan, versican, tertican, perlecan, bikunin, neurocan, aggrecan, fibromodulin, or lumican. In an embodiment, the extracellular matrix may be included in an amount of 0.1 to 20 parts by weight based on 100 parts by weight of the polymer scaffold. The extracellular matrix may be included in an amount of, for example, 0.1 to 20 parts by weight, 0.1 to 15 parts by weight, 0.1 to 15 parts by weight, 1 to 20 parts by weight, 1 to 15 parts by weight, 5 to 20 parts by weight, 5 to 15 parts by weight, 5 to 10 parts by weight, or 10 to 20 parts by weight, based on 100 parts by weight of the polymer scaffold. In this regard, when the amount of the extracellular matrix is less than the above-described range, the biocompatibility improving effect may not be sufficiently obtained, and when the amount of the extracellular matrix exceeds the above-described range, mechanical properties of the polymer scaffold may deteriorate.

In another embodiment, the extracellular matrix may be decellularized. After culturing tissues or cells, the extracellular matrix may be decellularized by a physical or chemical method. The physical decellularization method may be, for example, a freeze-thaw method, sonication, and physical agitation, and the chemical decellularization method may be, for example, a method of treating animal-derived tissue powder with a hypotonic fluid such as water, an anionic surfactant, a nonionic surfactant, or a cationic surfactant, DNase, RNase, trypsin, or the like. In the chemical decellularization method, a Tris-HCl solution (pH 8.0) may be used as the hypotonic fluid, and sodium dodecyl sulfate (SDS), sodium deoxycholate, Triton X-200, and the like may be used as the anionic surfactant. In addition, Triton X-100, Tween 20, or Tween 80 may be used as the nonionic surfactant, and CHAPS, sulfobetaine-10 (SB-10), sulfobetaine-16 (SB-16), or tri-n-butyl phosphate, N-lauroyl-sarcosinate, IGEPAL CA-630, and the like may be used as the cationic surfactant. In addition, the decellularization may be performed after collecting tissue, e.g., kidney tissue, of an animal, and before or after performing a powdering process or simultaneously therewith.

The bioactive material may be a DNA fragment mixture or extracellular vesicles. In an embodiment, as a result of implanting the biodegradable polymer scaffold including the nanoceramic particles, the extracellular matrix, the DNA fragment mixture, the extracellular vesicle, and the biodegradable polymer into mice of a kidney injury model, regeneration of injured kidney tissue was confirmed because tissue regeneration ability was improved and expression of regeneration-associated factors increased. In addition, it was confirmed that glomerular regeneration increased and glomerulosclerosis decreased in the mice, and glomerular filtration rate (GFR) was recovered to a level similar to that of normal mice. Therefore, the biodegradable polymer scaffold according to an embodiment may have outstanding effects not only on regeneration of injured tissue but also on restoration of the functions by including the bioactive material such as the DNA fragment mixture and/or the extracellular vesicle.

The DNA fragment mixture may be included in an amount of 3 to 30 parts by weight based on 100 parts by weight of the polymer scaffold. The DNA fragment mixture may be included, for example, in an amount of 3 to 30 parts by weight, 3 to 25 parts by weight, 3 to 20 parts by weight, 5 to 30 parts by weight, 5 to 25 parts by weight, 5 to 20 parts by weight, 10 to 30 parts by weight, or 15 to 20 parts by weight based on 100 parts by weight of the polymer scaffold. In this case, when the amount of the DNA fragment mixture is less than the above-described range, bioactivity effect cannot be sufficiently obtained. When the amount of the DNA fragment mixture is greater than the above-described range, cytotoxicity may be induced. In addition, the DNA fragment mixture may be, for example, a nucleotide polymer including polynucleotides (PNs), polydeoxyribonucleotides (PDRNs), and hydrolyzed DNAs.

The extracellular vesicle is a nano-sized vesicle secreted by all cells to the external environment for exchange of information between cells and includes various substances having bioactivity such as proteins, lipids, nucleic acids, and metabolites. The extracellular vesicle may include exosomes and microvesicles. The exosome may be isolated from stem cells derived from umbilical cord, cord blood, bone marrow, fat, muscle, skin, amnion, and placenta.

The biodegradable polymer may be at least one selected from the group consisting of poly-L-lactide, poly-D-lactide, poly-D, L-lactide, polyglycolide, polycaprolactone, poly-L-lactide-co-glycolide, poly-D-lactide-co-glycolide, poly-D,L-lactide-co-glycolide, poly-L-lactide-co-caprolactone, poly-D-lactide-co-caprolactone, poly-D,L-lactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyphosphazene, polyanhydride, polysebacic anhydride, polyhydroxyalkanoate, polyhydroxybutylate, polycyanoacrylate.

According to another aspect of the present disclosure, a biomedical implant including the biodegradable polymer scaffold is provided. Detailed descriptions of the biodegradable polymer scaffold are as given above.

In an embodiment, the surface of the biomedical implant may be modified or coated with the biodegradable polymer scaffold. The biomedical implant may be, for example, a scaffold for tissue regeneration, a stent, a surgical suture, a bio nanofiber, a hydrogel, a bio-sponge, a pin, a screw, a rod, an implant, or the like.

According to another aspect of the present disclosure, a method of manufacturing a biodegradable polymer scaffold includes preparing basic ceramic nanoparticles.

In an embodiment, the method may include: preparing basic ceramic nanoparticles; preparing a first polymer solution including the basic ceramic nanoparticles, an extracellular matrix, and a biodegradable polymer; preparing a second polymer solution by mixing 100 to 2000 parts by weight of a pore inducer inducing pores having a size of 100 to 500 μm with 100 parts by weight of the first polymer solution; and preparing a porous polymer scaffold by freeze-drying the second polymer solution. Detailed descriptions of the basic ceramic nanoparticles, the extracellular matrix, the DNA fragment mixture, and the biodegradable polymer are as given above. The preparing of the first polymer solution may include mixing the basic ceramic nanoparticles, the extracellular matrix, and the biodegradable polymer in an organic solvent. The organic solvent may be: for example, an alcohol such as methanol, ethanol, propanol, and butanol; an aldehyde such as ammonia, dimethyl sulfoxide, dimethyl formamide, acetronitrile, tetrahydrofuran, formaldehyde, glutaraldehyde, and acetaldehyde; an alkane such as dioxane, chloroform, heptane, hexane, pentane, octane, nonane, and decane; a benzene ring-type solvent such as benzene, toluene, and xylene; an ether such as ether, di-propyl ether, petrolium ether, and methyl-t-butyl ether; a ketone such as propanone, butanone, pentanone, hexanone, and heptanone; and a common organic solvent such as methylene chloride, tetrafluoroisopropane, and carbon tetrachloride.

In another embodiment, the first polymer solution may further include a DNA fragment mixture.

The pore inducer may be ice particles. By adjusting a size and content of the ice particles, a pore size and porosity of the porous polymer scaffold may be controlled. The size of the ice particles may be from 10 to 500 μm. The size of the ice particles may be, for example, from 10 to 500 μm, from 10 to 450 μm, from 10 to 400 μm, from 10 to 300 μm, from 30 to 500 μm, from 30 to 300 μm, from 30 to 250 μm, from 50 to 500 μm, from 50 to 400 μm, from 50 to 300 μm, from 50 to 200 μm, from 100 to 500 μm, from 100 to 300 μm, or from 150 to 200 μm. In this regard, when the size of the ice particles is less than the above-described ranges, a degree of cellular infiltration is too low to obtain sufficient angiogenesis effect. When the size of the ice particles is greater than the above-described ranges, mechanical properties of the polymer scaffold may deteriorate. In addition, the ice particles may be included in an amount of 100 to 2000 parts by weight based on 100 parts by weight of the first polymer basic solution. The ice particles may be included in an amount of 100 to 2000 parts by weight, 100 to 1500 parts by weight, 100 to 1300 parts by weight, 100 to 1000 parts by weight, 100 to 500 parts by weight, 500 to 2000 parts by weight, 500 to 1500 parts by weight, 500 to 1000 parts by weight, 1000 to 2000 parts by weight, or 1500 to 2000 parts by weight based on 100 parts by weight of the first polymer solution. In this case, when the amount of the ice particles is less than the above-described ranges, pores are not sufficiently formed in the polymer scaffold. When the amount of the ice particles is greater than the above-described ranges, mechanical properties of the polymer scaffold may deteriorate. In addition, by manufacturing the polymer scaffold using the ice particles by a freeze-dry method, separation of the basic ceramic nanoparticles and the bioactive material (the extracellular matrix and the DNA fragment mixture), which may occur in a common salt forming method, may be prevented.

The method may further include supporting extracellular vesicles on the porous polymer scaffold. Detailed descriptions of the extracellular vesicles are as given above. The extracellular vesicles may be supported on the porous polymer scaffold at a capacity of 10 to 500 μg by a simple loading method. The amount of the extracellular vesicles supported on the porous polymer scaffold may be, for example, from 10 to 500 μg, from 10 to 400 μg, from 10 to 300 μg, from 10 to 200 μg, from 50 to 500 μg, from 50 to 450 μg, from 50 to 350 μg, from 50 to 200 μg, from 100 to 500 μg, from 100 to 300 μg, or from 200 to 400 μg. In this regard, when the amount of the extracellular vesicles is less than the above-described ranges, the tissue regeneration effect may not be sufficiently obtained. When the amount is greater than the above-described ranges, the extracellular vesicles cannot be supported on the porous polymer scaffold because the amount exceeds a maximum loadable capacity.

### Advantageous Effects

The biodegradable polymer scaffold, preventing inflammatory responses caused by acidic substances produced during a degradation process, has easily controllable mechanical strength, and includes a bioactive material derived from cells of target tissues, and thus may induce tissue regeneration more effectively.

### DESCRIPTION OF DRAWINGS

FIG. 1 is a schematic diagram of a biodegradable polymer scaffold according to an embodiment.

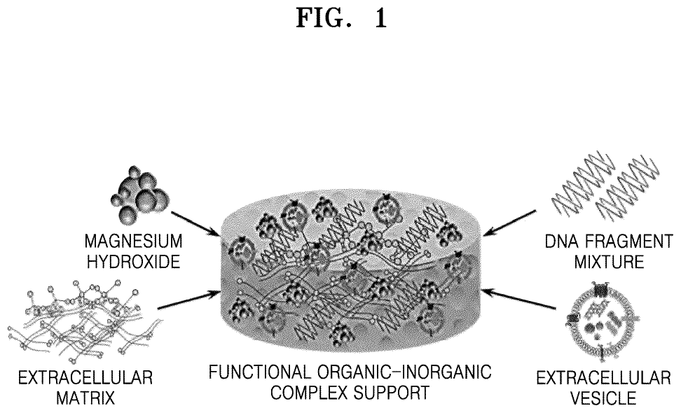

FIG. 2 is a graph showing particle sizes of basic ceramic particles according to an embodiment before and after surface modification.

FIG. 3A is a graph showing a compressive stress-strain curve of a biodegradable polymer scaffold according to an embodiment.

FIG. 3B is a graph showing the modulus of a biodegradable polymer scaffold according to an embodiment.

FIG. 3C is a graph showing degradation behavior of a biodegradable polymer scaffold according to an embodiment.

FIG. 3D is a graph showing pH change of a biodegradable polymer scaffold according to an embodiment.

FIG. 3E show images of pore size and structure of a biodegradable polymer scaffold according to an embodiment, obtained by using a scanning electron microscope (SEM).

FIG. 4A is a graph showing release behavior of a DNA fragment mixture of a biodegradable polymer scaffold according to an embodiment.

FIG. 4B shows SEM images of exosomes supported on a biodegradable polymer scaffold according to an embodiment.

FIG. 4C shows confocal laser SEM images showing fluorescence-stained exosomes supported on a biodegradable polymer scaffold according to an embodiment.

FIG. 5A is a graph showing growth rates of cells in a biodegradable polymer scaffold according to an embodiment.

FIG. 5B shows images for analyzing cytotoxicity in a biodegradable polymer scaffold according to an embodiment.

FIG. 6A shows histological staining images obtained by implanting a biodegradable polymer scaffold according to an embodiment into a partial nephrectomy mouse model and excising a kidney.

FIG. 6B is a graph showing the expression of regeneration-associated factors analyzed by real-time polymerase chain reaction after a biodegradable polymer scaffold according to an embodiment is implanted into a partial nephrectomy mouse model and a kidney is excised.

FIG. 7A is a graph showing the number of regenerated glomeruli after a biodegradable polymer scaffold according to an embodiment is implanted into a partial nephrectomy mouse model.

FIG. 7B is a graph showing regenerated glomerulosclerosis after a biodegradable polymer scaffold according to an embodiment is implanted into a partial nephrectomy mouse model.

FIG. 7C is a graph for analyzing the glomerular filtration rate after a biodegradable polymer scaffold according to an embodiment is implanted into a partial nephrectomy mouse model.

### MODE FOR INVENTION

Hereinafter, the present disclosure will be described in more detail with reference to the following examples. However, the following examples are merely presented to exemplify the present disclosure, and the scope of the present disclosure is not limited thereto.

### PREPARATION EXAMPLES

### Preparation Example 1. Preparation of Basic Ceramic Nanoparticles

### 1-1. Preparation of Magnesium Hydroxide Particles

10.8 g of sodium hydroxide was dissolved in 300 ml of deionized water to prepare a sodium hydroxide solution. Subsequently, a magnesium nitrate solution prepared by dissolving 20 g of magnesium nitrate in 150 ml of deionized water was added dropwise to the sodium hydroxide solution at a rate of 40 drops per minute using a dropping funnel. Magnesium hydroxide nanoparticles precipitated in the reaction solution were purified by flowing distilled water and the filtered. The obtained magnesium hydroxide nanoparticles were vacuum dried and stored.

### 1-2. Preparation of Magnesium Oxide Particles

The magnesium hydroxide particles prepared in Preparation Example 1-1 were calcined using an electric furnace at a temperature of 500 to 1500° C. to prepare magnesium oxide particles.

1-3. Preparation of Magnesium Hydroxide Particles Surface-Modified with Polymer

The basic ceramic nanoparticles prepared in Preparation Example 1-1 were surface-modified with L-lactide. Specifically, 80 parts by weight of magnesium hydroxide prepared in Preparation Example 1-1 and 20 parts by weight of L-lactide were mixed based on a total weight of the entire mixture. Subsequently, 0.05 wt % of tin octoate (catalyst) diluted in toluene was added to the reactants (magnesium hydroxide and L-lactide) based on the total weight of the reactants. A glass reactor containing the reactants was maintained at 70° C. in a vacuum state for 6 hours while stirring to completely remove toluene and moisture. The sealed glass reactor was stirred in an oil bath adjusted to 150° C. to perform ring-opening polymerization for 48 hours. A recovered polymer was placed in a sufficient amount of chloroform for over 1 hour to remove homopolymers and unreacted residues, thereby preparing basic ceramic nanoparticles modified with the polymer.

1-4. Preparation of Magnesium Oxide Particles Surface Modified with Polymer

Basic ceramic nanoparticles modified with the polymer was prepared in the same manner as in Preparation Example 1-3 except that the magnesium oxide particles prepared in Preparation Example 1-2 were used.

### Preparation Example 2. Preparation of Decellularized and Powdered Extracellular Matrix

2-1. Derived from Human Fat

Adipose tissue was collected from an individual and washed three times with a physiological saline solution for 10 minutes. After dehydrating the washed tissue with ethanol, adipocytes and genetic components present in the tissue were removed to obtain pure extracellular matrix by decellularization. Specifically, the dehydrated tissue was added to a 0.1% sodium dodecyl sulfate (SDS) solution at a rate of 10 g per 1 L, followed by stirring at 100 rpm for 24 hours. Subsequently, the resultant was washed five times with deionized water at 100 rpm for 30 minutes, and 200 ml of DNase having a concentration of 200 U/ml was added thereto, followed by stirring at 37° C. at 100 rpm for 24 hours. Then, the resultant was washed five times with deionized water at 100 rpm for 30 minutes and dried. The decellularized extracellular matrix was freeze-pulverized to a size of about 50 μm using a freezer mill to obtain powder.

2-2. Derived from Human Skin

An extracellular matrix was prepared in the same manner as in Preparation Example 2-1, except that skin tissue of an individual was used.

2-3. Derived from Pig Kidney

An extracellular matrix was prepared in the same manner as in Preparation Example 2-1, except that kidney tissue of a pig was used.

2-4. Derived from Mouse Kidney

An extracellular matrix was prepared in the same manner as in Preparation Example 2-1, except that kidney tissue of a mouse was used.

### Preparation Example 3. Isolation of Stem Cell-derived Exosome

Stem cells derived from human umbilical cord were proliferated by about 70% and washed twice with a phosphate buffer saline solution. Then, the medium was replaced with a phenol red-free medium containing 10% fetal bovine serum from which exosomes were removed. While the stem cells were cultured, supernatants were collected therefrom four times at every 12 hours. After removing impurities by filtering the culture media with a 0.22 μm filter, exosomes were selectively isolated and concentrated by using tangential flow filtration (TFF) with a MWCO 300 or 500 kDa filter.

### EXAMPLES

### Example 1. Bioactive Material-Containing Polymer Scaffold (1)

Exosomes were loaded on a porous polymer scaffold including basic ceramic nanoparticles, an extracellular matrix, extracellular vesicles, and a biodegradable polymer to prepare an exosome-loaded polymer scaffold. Specifically, based on a total weight of a polymer solution, 20 parts by weight of the basic ceramic nanoparticles of Preparation Example 1-1, 10 parts by weight of the extracellular matrix powder of Preparation Example 2-1, 20 parts by weight of polydeoxyribonucleotide (PDRN), and 50 parts by weight of a polylactide-co-glycolide (50:50) biodegradable polymer having a molecular weight of 40 K were mixed with an organic solvent to prepare a polymer solution. Then, ice particles with a size of 100 to 300 μm were added to the polymer solution in an amount of 1600 parts by weight based on a total weight of the polymer solution and uniformly mixed. Then, after fixing size and shape of a scaffold in a liquid nitrogen by using a silicone mold, the scaffold was freeze-dried under the conditions of 0° C. and 5 mTorr to prepare a porous polymer scaffold. Then, the porous polymer scaffold was sterilized by immersion in ethanol and washed with sterile distilled water to remove ethanol, and immersed in a physiological saline solution for hydration. After measuring an amount of proteins of exosomes isolated in Preparation Example 3 by the BCA color development assay, 100 μg of exosomes were loaded on the porous polymer scaffold by a simple loading method to prepare a bioactive material-containing polymer scaffold.

### Example 2. Bioactive Material-Containing Polymer Scaffold (2)

A bioactive material-containing polymer scaffold was prepared in the same manner as in Example 1, except that a polylactide-co-glycolide (50:50) biodegradable polymer having a molecular weight of 80 K was used.

### Example 3. Bioactive Material-Containing Polymer Scaffold (3)

A bioactive material-containing polymer scaffold was prepared in the same manner as in Example 1, except that the extracellular matrix powder of Preparation Example 2-3, polynucleotide (PN), and a polylactide-co-glycolide (50:50) biodegradable polymer having a molecular weight of 110 K were used.

### Example 4. Bioactive Material-Containing Polymer Scaffold (4)

A bioactive material-containing polymer scaffold was prepared in the same manner as in Example 1, except that the basic ceramic nanoparticles of Preparation Example 1-2, the extracellular matrix powder of Preparation Example 2-2, and a polylactide-co-glycolide (50:50) biodegradable polymer having a molecular weight of 80 K were used.

### Example 5. Bioactive Material-Containing Polymer Scaffold (5)

A bioactive material-containing polymer scaffold was prepared in the same manner as in Example 1, except that 20 parts by weight of the surface-modified basic ceramic nanoparticles of Preparation Example 1-3, 10 parts by weight of the extracellular matrix powder of Preparation Example 2-3, and 70 parts by weight of a polylactide-co-glycolide (85:15) biodegradable polymer having a molecular weight of 100 K were used.

### Example 6. Porous Polymer Scaffold (6)

A porous polymer scaffold was prepared in the same manner as in Example 1, except that the basic ceramic nanoparticles of Preparation Example 1-2, the extracellular matrix powder of Preparation Example 2-3, and the polylactide-co-glycolide (50:50) biodegradable polymer having a molecular weight of 110 K were used.

### Example 7. Porous Polymer Scaffold (7)

A porous polymer scaffold was prepared in the same manner as in Example 6, except that hydrolyzed DNA and the polylactide-co-glycolide (75:25) biodegradable polymer having a molecular weight of 100 K were used.

### Example 8. Porous Polymer Scaffold (8)

A porous polymer scaffold was prepared in the same manner as in Example 6, except that the basic ceramic nanoparticles of Preparation Example 1-3, the extracellular matrix powder of Preparation Example 2-4, and the poly-L-lactic acid biodegradable polymer having a molecular weight of 100 K were used.

### Example 9. Porous Polymer Scaffold (9)

A porous polymer scaffold was prepared in the same manner as in Example 1, except that 15 parts by weight of the surface-modified basic ceramic nanoparticles of Preparation Example 1-3, 10 parts by weight of the extracellular matrix powder of Preparation Example 2-2, and 75 parts by weight of a poly-L-lactic acid biodegradable polymer having a molecular weight of 270 K were used, and the DNA fragment mixture was not used.

### Example 10. Porous Polymer Scaffold (10)

A porous polymer scaffold was prepared in the same manner as in Example 9, except that the surface-modified basic ceramic nanoparticles of Preparation Example 1-3, the extracellular matrix powder of Preparation Example 2-4, and a polylactide-caprolactone (70:30) biodegradable polymer having a molecular weight of 168 K were used.

### Example 11. Porous Polymer Scaffold (11)

A porous polymer scaffold was prepared in the same manner as in Example 9, except that the surface-modified basic ceramic nanoparticles of Preparation Example 1-4, the extracellular matrix powder of Preparation Example 2-3, and a polylactide-caprolactone (70:30) biodegradable polymer having a molecular weight of 100 K were used.

### Comparative Example

### Comparative Example 1. Polymer Scaffold (1)

A polymer scaffold was prepared in the same manner as in Example 1, except that only the polylactide-co-glycolide (50:50) biodegradable polymer having a molecular weight of 40 K was used.

### Comparative Example 2. Polymer Scaffold (2)

A polymer scaffold was prepared in the same manner as in Example 1, except that 20 parts by weight of the basic ceramic nanoparticles of Preparation Example 1-1 and 80 parts by weight of the polylactide-co-glycolide (50:50) biodegradable polymer having a molecular weight of 40 K were used.

### Comparative Example 3. Polymer Scaffold (3)

A polymer scaffold was prepared in the same manner as in Example 1, except that 20 parts by weight of the basic ceramic nanoparticles of Preparation Example 1-1, 20 parts by weight of PDRN, and 60 parts by weight of the polylactide-co-glycolide (50:50) biodegradable polymer having a molecular weight of 40 K were used.

### Comparative Example 4. Bioactive Material-Containing Polymer Scaffold (4)

A bioactive material-containing polymer scaffold was prepared in the same manner as in Example 5, except that 20 parts by weight of the basic ceramic nanoparticles of Preparation Example 1-1, and 80 parts by weight of the polylactide-co-glycolide (50:50) biodegradable polymer having a molecular weight of 40 K were used.

### Comparative Example 5. Bioactive Material-Containing Polymer Scaffold (5)

A bioactive material-containing polymer scaffold was prepared in the same manner as in Example 1, except that 90 parts by weight of the polylactide-co-glycolide (50:50) biodegradable polymer having a molecular weight of 40 K, 10 parts by weight of the extracellular matrix of Preparation Example 2-1, and the exosomes of Preparation Example 3 were used.

### Comparative Example 6. Bioactive Material-Containing Polymer Scaffold (6)

A polymer scaffold loaded with exosomes was prepared in the same manner as in Example 1, except that 20 parts by weight of the basic ceramic nanoparticles of Preparation Example 1-1, 20 parts by weight of polydeoxyribonucleotide (PDRN), 60 parts by weight of the polylactide-co-glycolide (50:50) biodegradable polymer having a molecular weight of 40 K, and the exosomes of Preparation Example 3 were used.

### EXPERIMENTAL EXAMPLE

### Experimental Example 1. Identification of Size of Basic Ceramic Nanoparticles

Sizes of the basic ceramic nanoparticles prepared in Preparation Example 1 were identified by using a nanoparticle analyzer or a particle size analyzer (Malvern Zen 3600 Zatasizer, Zetasizer Ivano, UK).

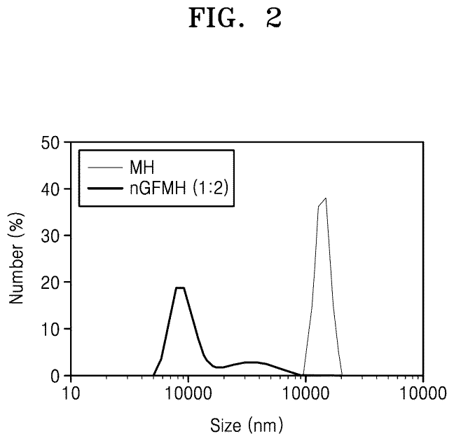

FIG. 2 is a graph showing particle sizes of basic ceramic particles according to an embodiment before and after surface modification.

As a result, as shown in FIG. 2, it was confirmed that the basic ceramic nanoparticles of Preparation Example 1-1 had an average particle size of 2.382 μm, and those of Preparation Example 1-3 surface-modified with the polymer had an average particle size of 0.48 μm. That is, the basic ceramic nanoparticles surface-modified with the polymer according to an embodiment may have improved dispersity by surface modification.

### Experimental Example 2. Measurement of Mechanical Tensile Strength of Polymer Scaffold

Mechanical tensile strengths of the polymer scaffolds prepared in Example 1 and Comparative Examples 1 to 4 were measured according to a method regulated by ASTM D638 by an Instron tester.

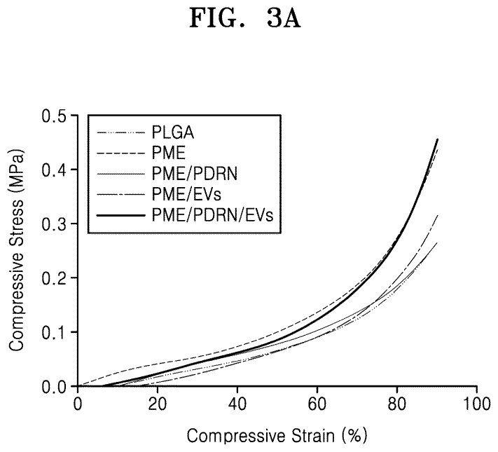

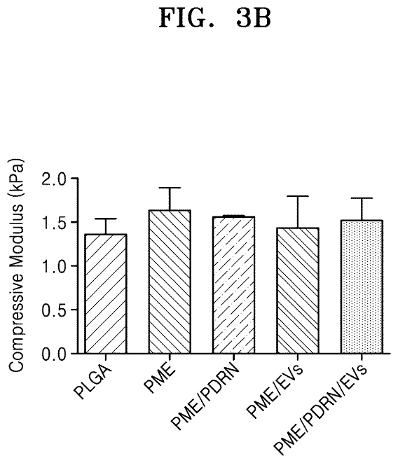

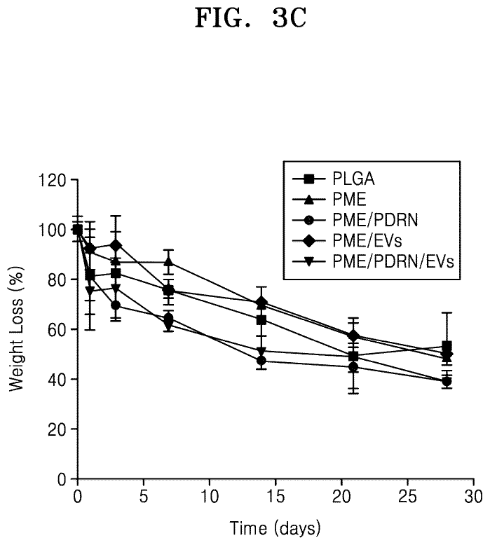

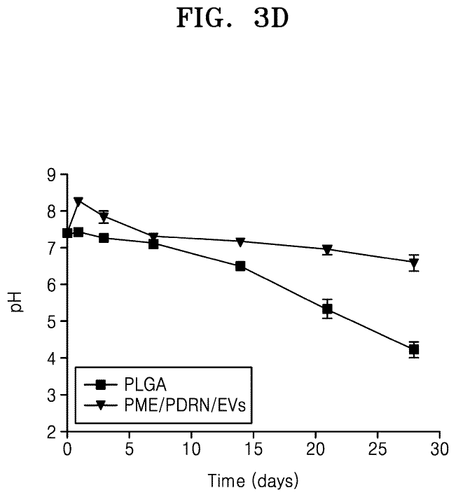

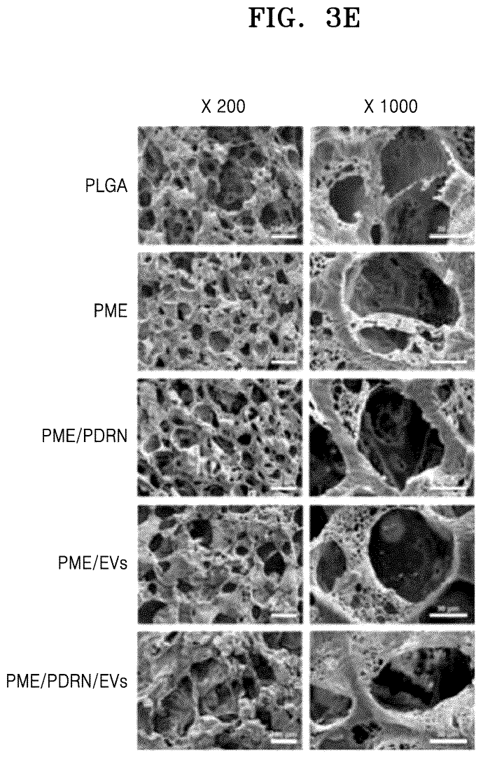

FIG. 3A is a graph showing a compressive stress-strain curve of a biodegradable polymer scaffold according to an embodiment.

FIG. 3B is a graph showing compressive modulus of a biodegradable polymer scaffold according to an embodiment.

As a result, as shown in FIGS. 3A and 3B, it was confirmed that the polymer scaffold of Example 1 had improved mechanical properties in comparison with that of Comparative Example 1. That is, the polymer scaffold according to an embodiment may have improved mechanical properties by including the basic ceramic nanoparticles, the extracellular matrix, and the bioactive materials.

### Experimental Example 3. Identification of Change in Water Contact Angle of Polymer Scaffold

Water contact angles of the polymer scaffolds prepared in Example 1 and Comparative Examples 1 to 3 were measured and the results are shown in Table 1 below.

TABLE 1

| | Water contact angle (deg) |
|---|---|
| Example 1 | Wetting |
| Comparative Example 1 | 115.72 ± 5.40 |
| Comparative Example 2 | 92.93 ± 2.02 |
| Comparative Example 3 | 93.45 ± 2.10 |

As a result, as shown in Table 1, it was confirmed that the polymer scaffold of Example 1 had a water contact angle far lower than those of Comparative Examples 1 to 3 and had improved hydrophilicity by supporting exosomes.

### Experimental Example 4. Identification of Biodegradation Duration and pH Change of Polymer Scaffold

Biodegradation durations and pH changes of the polymer scaffolds prepared in Example 1 and Comparative Example 1 to 4 were identified.

FIG. 3C is a graph showing a degradation behavior of a biodegradable polymer scaffold according to an embodiment.

FIG. 3D is a graph showing a pH change of a biodegradable polymer scaffold according to an embodiment.

As a result, as shown in FIG. 3C, it was confirmed that the polymer scaffold of Example 1 had a significantly higher initial (0 to 15 days) degradation rate compared to Comparative Examples 1 to 4. In addition, as shown in FIG. 3D, it was confirmed that the polymer scaffold of Example 1 had a less significant pH change over time than that of Comparative Example 1.

### Experimental Example 5. Identification of Pore Size and Structure of Polymer Scaffold

Pore sizes and structures of the polymer scaffolds prepared in Example 1 and Comparative Examples 1 to 4 were analyzed by using a scanning electron microscope (SEM).

FIG. 3E show images of pore size and structure of a biodegradable polymer scaffold according to an embodiment obtained by using a SEM.

As a result, as shown in FIG. 3E, an average pore size of the scaffold of Example 1 was measured as 200 μm. That is, the pore size of the polymer scaffold may be adjusted by controlling a size or amount of the pore inducer.

### Experimental Example 6. Identification of Release Behavior of Polymer Scaffold

A release behavior of the DNA fragment mixture of the polymer scaffold prepared in Example 1 was identified. Specifically, the polymer scaffold of Example 1 was immersed in 1 mL of nuclease-free water and stored for 28 days. After immersion, supernatants were collected on Day 1, Day 2, Day 3, Day 5, Day 7, and Day 28 and reacted with a DNA intercalator to measure and analyze fluorescence values.

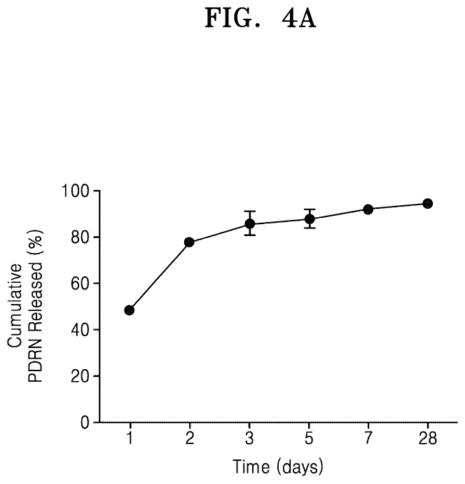

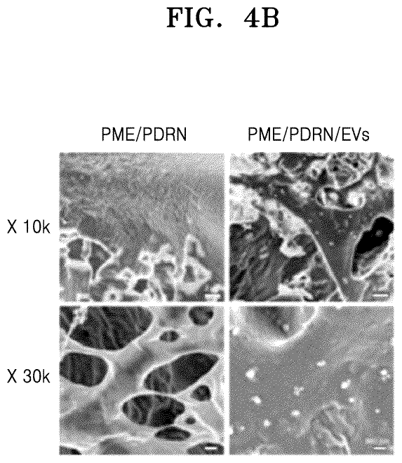

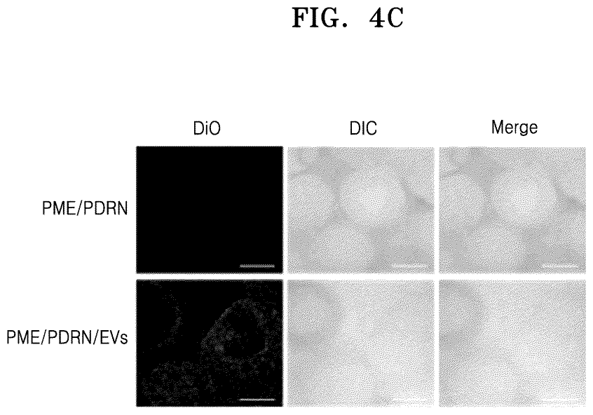

FIG. 4A is a graph showing a release behavior of a DNA fragment mixture of a biodegradable polymer scaffold according to an embodiment.

As a result, as shown in FIG. 4A, it was confirmed that about 47% of PDRN supported on the polymer scaffold of Example 1 was released after 24 hours from immersion, and about 95% of PDRN was released while the test was performed (28 days). That is, the biodegradable polymer scaffold according to an embodiment may deliver the DNA fragment mixture to target cells or tissues by effectively releasing the DNA fragment mixture supported on the scaffold.

### Experimental Example 7. Identification of Exosome Supported on Polymer Scaffold

By using a field emission type SEM, identified was whether exosomes supported on the polymer scaffolds of Example 1 and Comparative Example 3 were coated on the surfaces of the pores of the scaffolds. In addition, after staining the exosomes with a lipophilic fluorescent substance and simply loading the exosomes into the scaffold, the scaffold was observed by using a confocal laser SEM microscope.

FIG. 4B shows SEM images of exosomes supported on a biodegradable polymer scaffold according to an embodiment.

FIG. 4C shows confocal laser SEM images showing exosomes fluorescence-stained and supported on a biodegradable polymer scaffold according to an embodiment.

As a result, as shown in FIGS. 4B and 4C, although exosomes were not observed in Comparative Example 3, uniform distribution of exosomes was confirmed inner and outer surfaces of the polymer scaffold of Example 1.

### Experimental Example 8. Identification of Cytotoxicity of Scaffold

In order to evaluate cytotoxicity of the polymer scaffolds prepared in Example 1 and Comparative Examples 1 to 4, kidney-derived cells were cultured on the scaffolds of Example 1 and Comparative Examples 1 to 3 and 5 and cell growth rates and cytotoxicity were analyzed by staining with CCK-8 and Calcein AM/EthD-1, respectively.

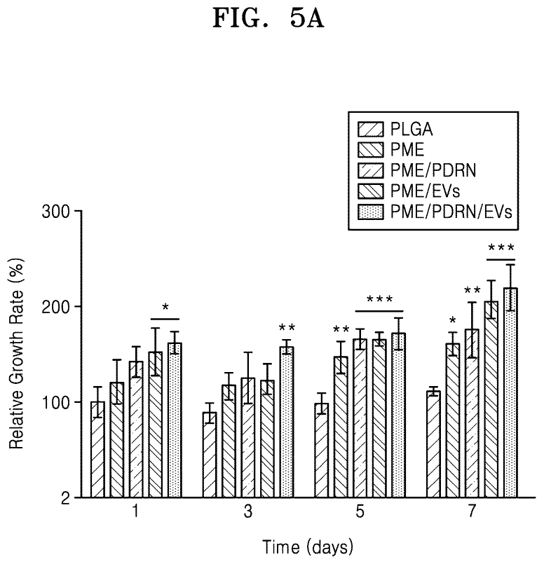

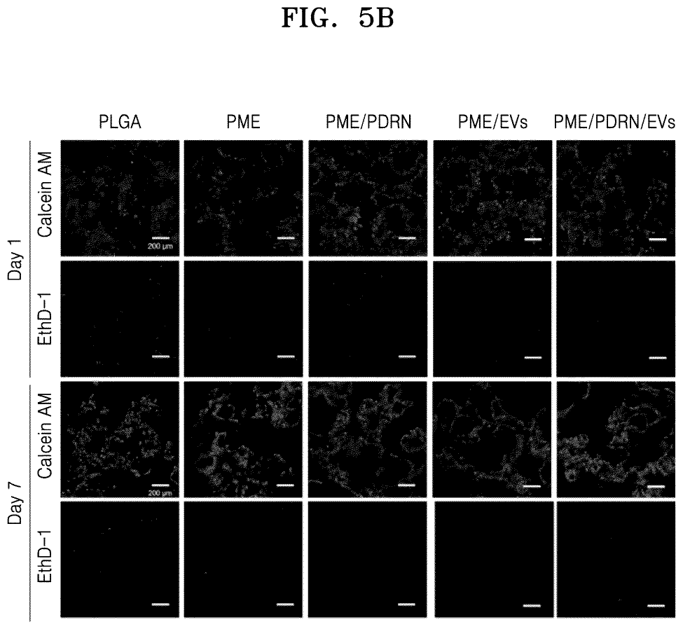

FIG. 5A is a graph showing growth rates of cells in a biodegradable polymer scaffold according to an embodiment.

FIG. 5B shows images for analyzing cytotoxicity in a biodegradable polymer scaffold according to an embodiment.

As a result, as shown in FIG. 5A, it was confirmed that the polymer scaffold of Example 1 exhibited a significantly high cell growth rate relative to those of Comparative Examples 1 to 4. Particularly, it was confirmed that the cell growth rate on Day 7 was about twice to three times that on Day 1. In addition, as shown in FIG. 5B, it was confirmed that the polymer scaffold of Example 1 exhibited higher fluorescence intensity and fluorescence was uniformly distributed compared to those of Comparative Examples 1 to 4. That is, the polymer scaffold according to an embodiment may effectively promote the growth of cells in the scaffold without cytotoxicity.

### Experimental Example 9. Evaluation of Tissue Regeneration Ability of Polymer Scaffold

In order to evaluate tissue regeneration ability of the polymer scaffolds of Example 1 and Comparative Examples 1 to 4, the scaffolds were implanted into a kidney injury model. Specifically, the polymer scaffolds prepared in Example 1 and Comparative Examples 1 to 4 were sterilized by using ethylene oxide gas and ethanol and immersed in a physiological saline solution. Mice of the kidney injury model were prepared by partial nephrectomy. After nephrectomy was performed on the mice of the kidney injury model, the polymer scaffold with a size of 5×2×2 m³ was implanted into a partially injured area of kidney cortex and maintained for 8 weeks. After 2 weeks and 8 weeks from the implantation, kidney tissue of the mice was excised and subjected to histological analysis by using immunohistochemical staining and polymerase chain reaction.

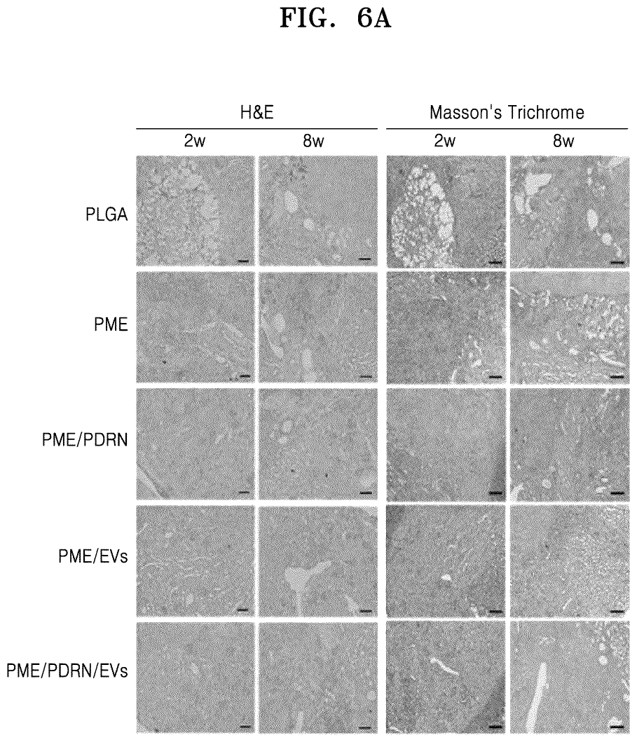

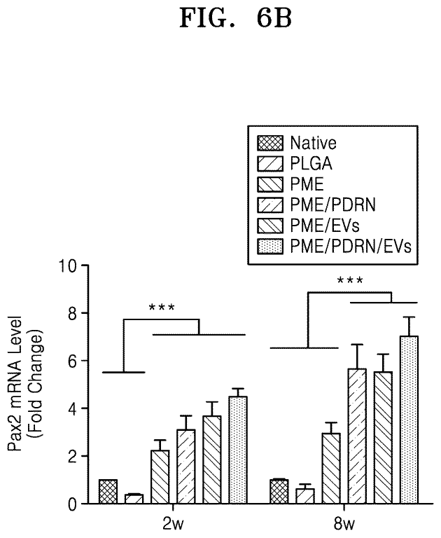

FIG. 6A shows histological staining images obtained by implanting a biodegradable polymer scaffold according to an embodiment into a partial nephrectomy mouse model and excising a kidney.

FIG. 6B is a graph showing expression of regeneration-associated factors analyzed by real-time polymerase chain reaction after a biodegradable polymer scaffold according to an embodiment is implanted into a partial nephrectomy mouse model and a kidney is excised.

As a result, as shown in FIG. 6A, Comparative Examples 1 to 4 exhibited not only low tissue regeneration ability but also fibrosis of surrounding tissues. However, it was confirmed that the polymer scaffold of Example 1 had improved tissue regeneration ability and fibrosis of surrounding tissues was also significantly reduced. In addition, as shown in FIG. 6B, it was confirmed that the expression level of Pax2 mRNA, which is a kidney regeneration-associated factor, of Example 1 was significantly higher than those of Comparative Examples 1 to 4. Particularly, the expression level of Pax2 mRNA of the mice to which the scaffold of Example 1 was implanted was about 6 times higher than that of Comparative Example 1. That is, it was confirmed that the biodegradable polymer scaffold according to an embodiment had excellent tissue regeneration ability by including the bioactive materials such as the extracellular vesicles and the DNA fragment mixture.

### Experimental Example 10. Evaluation of Effect of Polymer Scaffold on Improving Kidney Function

The effects of the polymer scaffolds prepared in Example 1 and Comparative Examples 1 to 4 on improving functions of kidney were evaluated. Specifically, the number of regenerated glomeruli and the degree of glomerulosclerosis of the mice into which the polymer scaffold of Experimental Example 8 was implanted were analyzed by histological staining, and glomerular filtration rates were analyzed by using FITC-inulin.

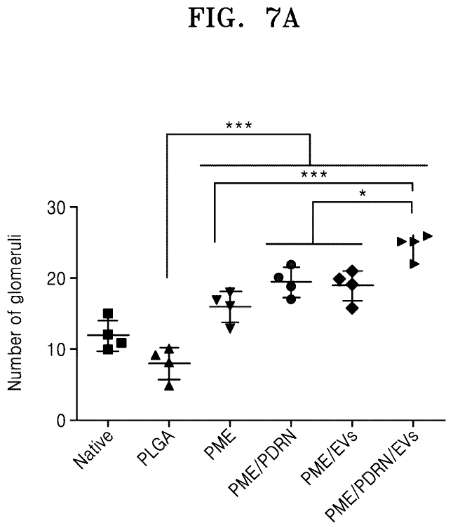

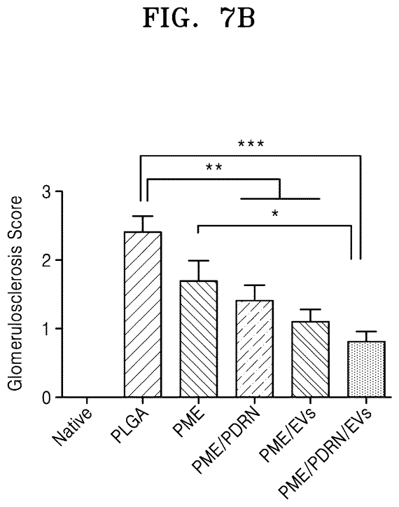

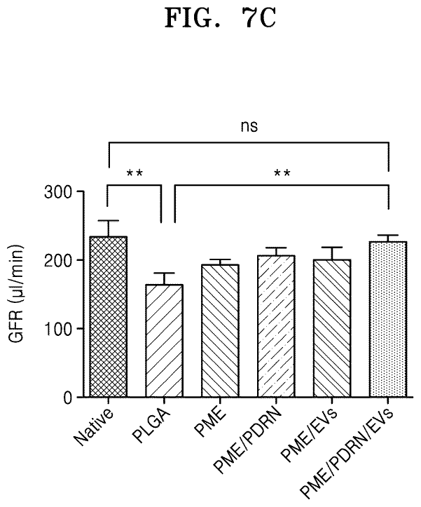

FIG. 7A is a graph showing the number of regenerated glomeruli after a biodegradable polymer scaffold according to an embodiment is implanted into a partial nephrectomy mouse model.

FIG. 7B is a graph showing regenerated glomerulosclerosis after a biodegradable polymer scaffold according to an embodiment is implanted into a partial nephrectomy mouse model.

FIG. 7C is a graph for analyzing glomerular filtration rates after a biodegradable polymer scaffold according to an embodiment is implanted into a partial nephrectomy mouse model.

As a result, as shown in FIG. 7A, it was confirmed that the number of regenerated glomeruli was significantly increased in the mice to which the scaffold of Example 1 was implanted compared with Comparative Examples 1 to 4. In addition, as shown in FIG. 7B, it was confirmed that the glomerulosclerosis score of the mice to which the scaffold of Example 1 was implanted was significantly low compared to the mice to which the scaffolds of Comparative Examples 1 to 4 were implanted, particularly, glomerulosclerosis score of the mice of Example 1 was decreased by about 50% or more compared to Comparative Example 1. In addition, as shown in FIG. 7C, it was confirmed that the glomerular filtration rates of mice to which the scaffold of Example 1 was implanted was recovered to a level similar to that of a normal control (Native). That is, the biodegradable polymer scaffold according to an embodiment promotes restoration of the functions of the injured kidney by including the bioactive materials, and thus may be efficiently used for tissue regeneration.

The above description of the present disclosure is provided for the purpose of illustration, and it would be understood by those skilled in the art that various changes and modifications may be made without changing technical conception and essential features of the present disclosure. Thus, it is clear that the above-described embodiments of the present disclosure are illustrative in all aspects and do not limit the present disclosure.

1. A biodegradable polymer scaffold comprising basic ceramic nanoparticles, an extracellular matrix, bioactive materials, and a biodegradable polymer.

2. The biodegradable polymer scaffold of claim 1, wherein the basic ceramic nanoparticles are: an alkali metal or an oxide or hydroxide thereof; or an alkali earth metal or an oxide or hydroxide thereof.

3. The biodegradable polymer scaffold of claim 2, wherein the alkali metal or alkali earth metal is selected from lithium (Li), beryllium (Be), sodium (Na), magnesium (Mg), potassium (K), calcium (Ca), rubidium (Rb), strontium (Sr), barium (Ba), cesium (Cs), francium (Fr), and radium (Ra).

4. The biodegradable polymer scaffold of claim 2, wherein the oxide or hydroxide of the alkali metal or alkali earth metal is selected from lithium hydroxide, beryllium hydroxide, sodium hydroxide, magnesium hydroxide, potassium hydroxide, calcium hydroxide, rubidium hydroxide, strontium hydroxide, barium hydroxide, cesium hydroxide, francium hydroxide, radium hydroxide, magnesium oxide, sodium oxide, lithium oxide, sodium oxide, manganese oxide, potassium oxide, calcium oxide, barium oxide, cesium oxide, and radium oxide.

5. The biodegradable polymer scaffold of claim 1, wherein surfaces of the basic ceramic nanoparticles are modified with fatty acids, biodegradable polymer materials, or a mixture thereof.

6. The biodegradable polymer scaffold of claim 1, wherein the bioactive material are DNA fragment mixtures, extracellular vesicles, or mixtures thereof.

7. The biodegradable polymer scaffold of claim 6, wherein the DNA fragment mixtures are selected from polynucleotide (PN), polydeoxyribonucleotide (PDRN), and hydrolyzed DNA.

8. The biodegradable polymer scaffold of claim 6, wherein the extracellular vesicles are exosomes, microvesicles, or a mixture thereof.

9. The biodegradable polymer scaffold of claim 6, wherein the extracellular vesicles are isolated from stem cells derived from umbilical cord, cord blood, bone marrow, fat, muscle, skin, amnion, or placenta.

10. The biodegradable polymer scaffold of claim 1, wherein the biodegradable polymer is selected from polylactide, polyglycolide, polycaprolactone, polylactide-co-glycolide, polylactide-co-caprolactone, polyglycolide-co-caprolactone, polydioxanone, polytrimethylene carbonate, polyglycolide-co-dioxanone, polyamide ester, polypeptide, polyorthoester, polymaleic acid, polyanhydride, polysebacic anhydride, polyhydroxyalkanoate, polyhydroxybutylate, and polycyanoacrylate.

11. The biodegradable polymer scaffold of claim 1, wherein the basic ceramic nanoparticles have a size of 1 nm to 1 mm.

12. A biomedical implant comprising the biodegradable polymer scaffold of claim 1.

13. The biomedical implant of claim 12, wherein a surface of the biomedical implant is modified or coated with the biodegradable polymer scaffold.

14. The biomedical implant of claim 12, wherein the biomedical implant is selected from a scaffold for tissue regeneration, a stent, a surgical suture, a bio nanofiber, a hydrogel, a bio-sponge, a pin, a screw, a rod, and an implant.

15. A method of manufacturing a biodegradable polymer scaffold, the method comprising: preparing basic ceramic nanoparticles;
preparing a first polymer solution including the basic ceramic nanoparticles, an extracellular matrix, and a biodegradable polymer;
preparing a second polymer solution by mixing 100 to 2000 parts by weight of a pore inducer inducing pores having a size of 100 to 500 μm, with 100 parts by weight of the first polymer solution; and
preparing a porous polymer scaffold by freeze-drying the second polymer solution.

16. The method of claim 15, wherein the first polymer solution further comprises a DNA fragment mixture.

17. The method of claim 15, further comprising loading extracellular vesicles into the porous polymer scaffold.

18. The method of claim 15, wherein the porous polymer scaffold further comprises a DNA fragment mixture and extracellular vesicles.
